(19) United States
(12) Patent Application Publication
(10) Pub. No.: US 2018/0256850 A1
(43) Pub. Date: Sep. 13, 2018

(54) MULTICOAXIAL CABLE

(71) Applicant: SUMITOMO ELECTRIC INDUSTRIES, LTD., Osaka (JP)

(72) Inventor: Tatsunori HAYASHISHITA, Hachinohe-shi (JP)

(73) Assignee: SUMITOMO ELECTRIC INDUSTRIES, LTD., Osaka (JP)

(21) Appl. No.: 15/884,731

(22) Filed: Jan. 31, 2018

(30) Foreign Application Priority Data
Feb. 2, 2017 (JP) 2017-017565

(51) Int. Cl. A61M 25/00; A61L 29/04; A61L 29/02; A61L 29/08; A61B 1/00; F16L 11/10; B32B 1/08; B32B 15/08

(52) U.S. Cl. CPC A61M 25/0045; A61L 29/049; A61L 29/02; A61L 29/085; A61B 1/00064; A61M 25/005; B32B 2535/00; B32B 1/08; B32B 15/08; A61M 2025/0046; B32B 2255/26; B32B 2255/10; F16L 11/10

(57) ABSTRACT

A multilayered tube having a lumen, in order from an inner side in a radial direction based on the central axis of the lumen, comprises an inner layer made of a resin, a metal layer which is disposed on an outer circumference of the inner layer and has a braided or spirally wrapped structure, an outer layer which covers the inner layer and the metal layer and is made of a resin, and a coating layer which is provided on the outer layer and is made of a substance different from the outer layer. An outer diameter of the multilayered tube is 1.5 mm or less.

### CROSS-REFERENCE TO RELATED APPLICATION

This application claims priority from Japanese Patent Application No. 2017-017565, filed on Feb. 2, 2017, the entire subject matter of which is incorporated herein by reference.

### TECHNICAL FIELD

The present invention relates to a multilayered tube for catheters or endoscopes.

### BACKGROUND

JP-A-2016-152907 discloses a catheter has a multilayered structure in which, in order from the inner side in a radial direction based on the central axis of a catheter main body, an inner layer, a braid, and an outer layer are included.

### SUMMARY

In general, as a tube for catheters or endoscopes, a tube is used which is obtained by extrusion molding or injection molding using silicone, polyurethane, PTFE or the like. The single-layered tube is required to have a certain thickness from the view of securing strength and a flexibility, and thus it is difficult to make a wall of the tube thin. Further, from the view of an operability at the time of being inserted into the body (for example, into a blood vessel), the tube is required to have a slipping property and not to be permeable to water.

An object of the present invention is to provide a multilayered tube which has thin wall, has a small diameter, and is further excellent in slipping property and not permeable to water.

An embodiment of the present invention is a multilayered tube having a lumen, in order from an inner side in a radial direction based on the central axis of the lumen, comprises an inner layer made of a resin, a metal layer which is disposed on an outer circumference of the inner layer and has a braided or spirally wrapped structure, an outer layer which covers the inner layer and the metal layer and is made of a resin, and a coating layer which is provided on the outer layer and is made of a substance different from the outer layer. An outer diameter of the multilayered tube is 1.5 mm or less.

According to the embodiment of the present invention, it is possible to provide a multilayered tube which has thin wall, has a small diameter, and is further excellent in slipping property and not permeable to water.

### BRIEF DESCRIPTION OF THE DRAWINGS

FIG. 1 is a cross-sectional view illustrating an example of a multilayered tube according to an embodiment of the present invention; and

FIG. 2 is a cross-sectional view illustrating another example of a multilayered tube according to the embodiment of the present invention.

### DETAILED DESCRIPTION

### Embodiment of Invention

First, the contents of an embodiment of the present invention will be listed below.

A multilayered tube according to the embodiment of the present invention of this application is characterized as follows.

(1) A multilayered tube having a lumen, in order from an inner side in a radial direction based on the central axis of the lumen, including:

an inner layer made of a resin;

a metal layer which is disposed on an outer circumference of the inner layer and has a braided or spirally wrapped structure;

an outer layer which covers the inner layer and the metal layer and is made of a resin; and

a coating layer which is provided on the outer layer and is made of a substance different from the outer layer,

wherein an outer diameter is 1.5 mm or less.

According to this configuration, it is possible to provide a multilayered tube which has thin wall, has a small diameter, and is further excellent in slipping property and not permeable to water.

(2) The multilayered tube according to (1),

wherein the ratio of the outer diameter and an inner diameter may be 1.15 or more and 1.4 or less in outer diameter/inner diameter.

According to this configuration, it is possible to provide a multilayered tube which has thin wall but has a size sufficient to accommodate an electric wire or the like in the tube and a sufficient strength.

(3) The multilayered tube according to (1) or (2),

wherein the inner layer may be formed of a resin tape to be spirally wrapped.

According to this configuration, it is possible to provide a multilayered tube which is further thin and has a smaller diameter.

(4) The multilayered tube according to any one of (1) to (3), further including:

a braided chemical fiber which is disposed on an outer circumference of the metal layer and is covered with the outer layer.

According to this configuration, it is possible to provide a multilayered tube which has thinner wall and has a smaller diameter but is excellent in durability.

### DETAILED DESCRIPTION OF EMBODIMENT

Next, an embodiment relating to a multilayered tube according to the present invention will be described with reference to the accompanying drawings.

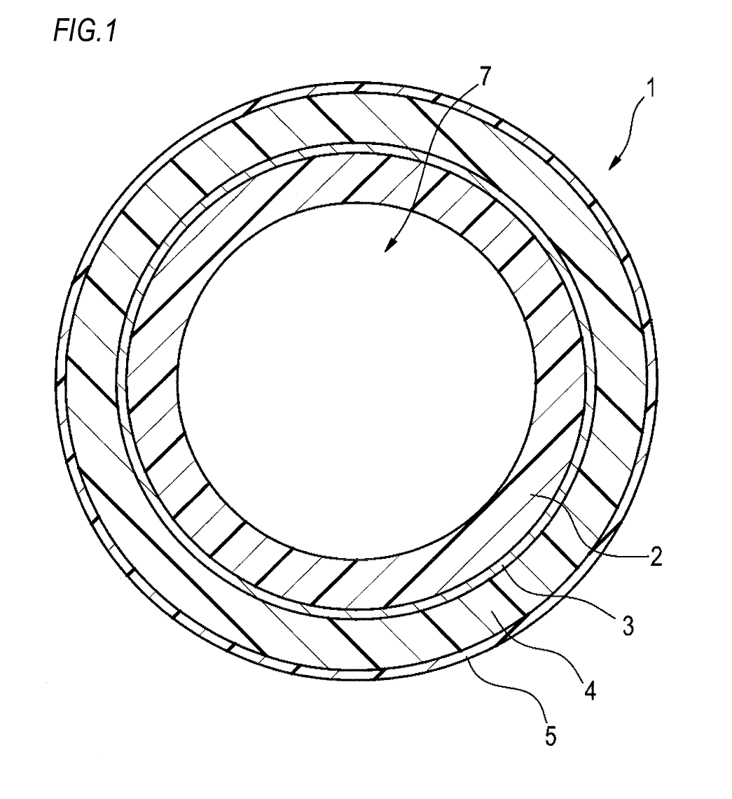

FIG. 1 is a cross-sectional view illustrating an example of a multilayered tube according to the embodiment of the present invention. In the drawing, a reference numeral “1” denotes the multilayered tube, a reference numeral “2” denotes an inner layer, a reference numeral “3” denotes a metal layer, a reference numeral “4” denotes an outer layer, a reference numeral “5” denotes a coating layer, and a reference numeral “7” denotes a lumen.

More specifically, the multilayered tube 1 illustrated in FIG. 1 has a tubular structure having the lumen 7, and includes the inner layer 2, the metal layer 3, the outer layer 4, and the coating layer 5 in order from the inner side in a radial direction based on the central axis of the lumen 7.

The outer diameter of the multilayered tube 1 is 1.5 mm or less. Further, the inner diameter of the multilayered tube 1 preferably has a size in which the outer diameter/inner diameter is 1.4 or less. Accordingly, it is possible to provide a multilayered tube that is thin but has a sufficient size to accommodate electric wires and the like in the tube. Further, the outer diameter/inner diameter is preferably 1.15 or more. Accordingly, it is possible to provide a multilayered tube that has thin wall and has sufficient strength.

The inner layer 2 is made of a resin. As the resin constituting the inner layer 2, it is preferable to use a resin having a good slipping property, for example, a thermoplastic resin such as a polyester resin is used. The thickness of the inner layer can be set to 0.01 to 0.03 mm. Also, a tape-shaped resin can be used, for example, a polyester film may be used. Since the thickness of the layer obtained by spirally wrapping the polyester film around the core material can be thinner than the thickness of the layer produced by usual extrusion molding, it is preferable to use a film for the inner layer. The winding pitch of the film can be set to 5 to 15 times the inner diameter of the tube.

The metal layer 3 is provided on the outer circumference of the inner layer 2. Examples of the metal constituting the metal layer 3 include stainless steel (SUS), copper, or an alloy such as a copper alloy, and the like. The metal layer may be plated with these metals. The metal layer 3 is formed by braiding or spirally wrapping the thin wire of these metals on the outer circumference of the inner layer 2. The diameter of the thin metal wire constituting the metal layer 3 is preferably in a range of 0.016 mm to 0.030 mm.

The outer layer 4 is formed to cover the inner layer 2 and the metal layer 3. The resin constituting the outer layer 4 is preferably a resin having a good slipping property, and it is preferable to use polyvinyl chloride (PVC), polyurethane (PUR), silicone, an acrylic resin or the like. The outer layer 4 is provided on the outer circumference of the inner layer 2 and the metal layer 3 by extrusion molding. The thickness of the outer layer can be set to 0.02 to 0.15 mm.

The coating layer 5 is provided on the outer layer 4. Examples of the material constituting the coating layer 5 include polyparaxylene, hyaluronic acid, or a fluororesin (PFA, ETFE, FEP, THV, or the like.), and the like. By providing the coating layer 5 made of these substances on the surface of the outer layer 4, it is possible to greatly improve the slipping property and prevent water from permeating. The thickness of the coating layer 5 is, for example, 0.5 μm or more and 3.0 μm or less.

In the multilayered tube 1, for example, a resin tape is spirally wrapped around the core material to form the inner layer 2, the metal layer 3 having a braided or spirally wrapped structure is provided on the outer circumference of the inner layer 2, the outer layer 4 made of a resin is provided on the outer circumference of the metal layer 3 by extrusion molding, and coating is performed on the outer layer 4. After the coating, the core material is pulled out, thereby producing the multilayered tube 1.

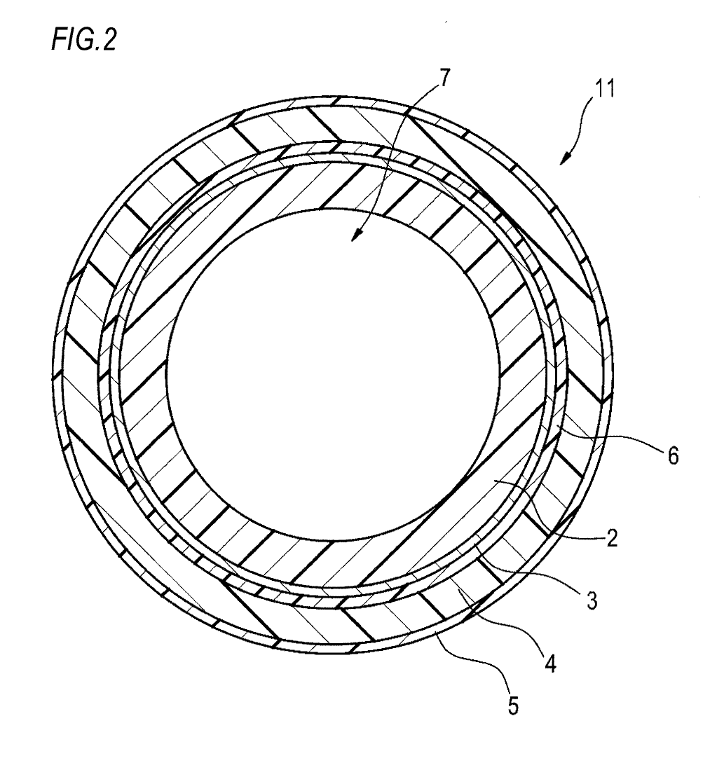

Although the embodiment of the present invention has been described with reference to FIG. 1, as a modification of this embodiment, a multilayered tube 11 may be applied which includes an inner layer 2, a metal layer 3, an outer layer 4, a coating layer 5, and further a braided chemical fiber 6 disposed on the outer circumference of the metal layer 3 and covered with the outer layer 4 as illustrated in FIG. 2. For the chemical fiber 6, a nylon thread or the like can be used. The thickness of the chemical fiber wire to be braided can be 80 to 120 deniers. The multilayered tube 11 has thin wall and a small diameter but excellent durability.

The multilayered tube according to the embodiment can be used for catheters and endoscopes. At that time, the length of the multilayered tube is set to about 50 cm, and an electric wire or the like can be passed through the multilayered tube.

### EXAMPLES

Next, the embodiment for carrying out the present invention will be described with Examples. The examples do not limit the scope of the present invention.

A polyester film was wrapped around the core material, a metal braid (obtained by braiding a stainless steel wire having a wire diameter of 0.025 mm) was provided thereon, and an acrylic resin was covered thereon by extrusion molding. Thereafter, the surface of the acrylic resin was coated with hyaluronic acid to remove the core material, thereby obtaining a multilayered tube of Example 1.

A polyester film was wrapped around the core material, a metal braid (similar to Example 1) was provided thereon, a braid of nylon threads was further provided thereon, and an acrylic resin was covered thereon by extrusion molding. Thereafter, the surface of the acrylic resin was coated with hyaluronic acid to remove the core material, thereby obtaining a multilayered tube of Example 2.

A polyester film was wrapped around the core material, a metal braid (similar to Example 1) was provided thereon, and an acrylic resin was covered thereon by extrusion molding. Thereafter, the core material was pulled out, and thereby obtaining a multilayered tube of Example 3.

Specific configurations of Examples 1 to 3 are as shown in Table 1. Using the multilayered tubes of Examples 1 to 3, the following slipping property test and water permeability test were carried out.

### Test of Slipping Property

The multilayered tube was placed on a resin film having the same material as the resin of the outer layer, applied with a load of 200 gram-force, and pulled at a speed of 100 mm/min in the longitudinal direction of the multilayered tube. At this time, a frictional force was measured and a static friction coefficient was evaluated. The multilayered tube was evaluated as “acceptable” in a case where the static friction coefficient is equal to or less than 0.5, or was evaluated as “unacceptable” in a case where the static friction coefficient was more than 0.5.

### Test of Water Permeability

After bending the multilayered tube into a U shape and immersing the bent multilayered tube by the length of 30 cm or more into warm water of 60 degrees for 189 hours, the multilayered tube was taken out from the warm water, and the immersed part was cut off. The inner surface of the immersed part of the multilayered tube was observed and evaluated as “acceptable” in a case where permeation of water was not recognized, or evaluated as “unacceptable” in a case where permeation of water was observed.

In Examples 1 and 2 provided with the coating layer, preferable results were obtained in the slipping property test and water permeability test. On the other hand, in Example 3 without the coating layer, preferable results were not obtained in the slipping property test and water permeability test.

TABLE 1

| | | | Example 1 | Example 2 | Example 3 |
|---|---|---|---|---|---|
| inner | material | | polyester | polyester | polyester |
| | | | film | film | film |
| layer | inner diameter (mm) | | 1.12 | 1.12 | 1.12 |
| | outer diameter (mm) | | 1.16 | 1.16 | 1.16 |
| braided | material | | steel wire | steel wire | steel wire |
| metal | wire diameter (mm) | | 0.025 | 0.025 | 0.025 |
| layer | structure | the number | 16 | 16 | 16 |
| | | of spindles | | | |
| | | the number | 10 | 10 | 10 |
| | | of ends | | | |
| | pitch (mm) | | 2.5 | 2.5 | 2.5 |
| braided | material | | none | nylon | none |
| resin | | | | thread | |
| layer | wire diameter (mm) | | | 0.01 | |
| | structure | the number | | 16 | |
| | | of spindles | | | |

| | | | | |
|---|---|---|---|---|
| | pitch (mm) | | 2.5 | |
| resin of | material | acrylic | acrylic | nylon |
| outer layer | | | | |
| coating | material | hyaluronic | hyaluronic | none |
| layer | | acid | acid | |

| | | | |
|---|---|---|---|
| outer diameter (mm) | 1.36 | 1.40 | 1.40 |
| slipping property | acceptable | acceptable | un- |
| | | | acceptable |
| water permeability | acceptable | acceptable | un- |
| | | | acceptable |

What is claimed is:

1. A multilayered tube having a lumen, in order from an inner side in a radial direction based on the central axis of the lumen, comprising:
an inner layer made of a resin;
a metal layer which is disposed on an outer circumference of the inner layer and has a braided or spirally wrapped structure;
an outer layer which covers the inner layer and the metal layer and is made of a resin; and
a coating layer which is provided on the outer layer and is made of a substance different from the outer layer,
wherein an outer diameter is 1.5 mm or less.

2. The multilayered tube according to claim 1,
wherein the ratio of the outer diameter and an inner diameter is 1.15 or more and 1.4 or less in outer diameter/inner diameter.

3. The multilayered tube according to claim 1,
wherein the inner layer is formed of a resin tape to be spirally wrapped.

4. The multilayered tube according to claim 1, further comprising:
a braided chemical fiber which is disposed on an outer circumference of the metal layer and is covered with the outer layer.
